(19) United States
(12) Patent Application Publication
(10) Pub. No.: US 2018/0187216 A1
(43) Pub. Date: Jul. 5, 2018

(54) USE OF MICROBACTERIUM STRAINS FOR THE PRODUCTION OF ANTIBACTERIAL AGENTS

(71) Applicant: DEINOBIOTICS, Grabels (FR)

(72) Inventors: Pascal DHULSTER, Lille (FR); Dominique LE BELLER, Jaux (FR); Jean-Paul LEONETTI, Grabels (FR); Huges MANDAVID, Grosbliederstroff (FR); Prémavathy LEVASSEUR, Enghien Les Bains (FR); Marjorie VALENDUC, Emmerin (FR)

(21) Appl. No.: 15/740,821

(22) PCT Filed: Jul. 1, 2016
(86) PCT No.: PCT/EP2016/065567

§ 371 Date: Dec. 29, 2017

(30) Foreign Application Priority Data
Jul. 1, 2015 (EP) 15306066.0

(51) Int. Cl. C12P 1/04; A01N 63/02

(52) U.S. Cl. CPC C12P 1/04; A01N 63/02

(57) ABSTRACT

The present invention relates to the use of a bacterium of the genus Microbacterium for the production of a compound endowed with antibacterial activity.

### BACKGROUND OF THE INVENTION

Antibacterial resistance, which entails the microorganisms ability to find ways aimed at circumventing the actions of the drugs used to cure the infections caused by such microorganisms, is held as a current public health issue not only because of the growing trend of resistant bacteria, but also due to the lack of new antibiotics.

Thus, there is a growing demand of antibiotics not only due to the resistance issue, but also to the extended life expectancy of the population.

For example, multi-drug resistant Gram-positive bacteria (MDRGP) still continue to pose challenges to the scientific community, which involve Staphylococcus aureus, whose first penicillin-resistant strains emerged more than fifty years ago. Also, the multiple-drug resistant Gram-negative bacteria (MDRGN) have turned into an issue of concern, particularly, the E.coli-resistant strains.

It has never been proved in the literature that strains belonging to the genus Microbacterium are capable to produce antibacterial compounds.

Learn-Han Lee et al. screened Actinobacteria isolates, among them Microbacterium spp. and Streptomyces spp., with the aim to identify those able to produce antimicrobial secondary metabolites (Learn-Han Lee et al., The Scientific World Journal, Vol. 6, No. 1, 1 Jan. 2014, p. 12-14). Only the Streptomyces spp. isolates exhibited activity against the pathogenic bacteria tested. The Microbacterium spp. isolates tested exhibit no antibacterial activity in the implemented conditions of isolation and fermentations.

Documents WO2010/081899 and US2014/017724 describe the use of a cell destructing DNA damaging treatment to isolate novel bacterial strains which produce active secondary metabolites. Among the isolated strains, it is shown that Microbacterium strain MA3-7G exhibited antifungal activity, but no activity against pathogenic bacteria was observed. Moreover, it can be noted that the used Microbacterium strain MA3-7G was obtained after irradiation of the corresponding natural strain, with no other comment.

Therefore, the search for new chemical entities endowed with antibacterial properties and the structures of which differ from those found in conventional antibiotics is viewed as a pressing need to develop new ways to curb these resistant infections. The applicant has found that Microbacterium is capable and particularly useful to produce novel compounds endowed with antibacterial activity. All Microbacterium strains described in the literature so far have been isolated from environmental sources. Initially, these yellow- or orange-pigmented, fermentative gram-positive rods (GPRs) were identified as CDC coryneform group A-4 and A-5 bacteria, but further investigations revealed that they belong to the genus Microbacterium (Primary Identification of Microbacterium spp. encountered in Clinical Specimens as CDC Coryneform Group A-4 and A-5 Bacteria, Guido FUNKE, JOURNAL OF CLINICAL MICROBIOLOGY, January 1995, p. 188-192).

### BRIEF SUMMARY OF THE INVENTION

The present invention relates to the use of a bacterium of the genus Microbacterium for the production of a compound endowed with antibacterial activity.

It has been shown that the genome of Microbacterium codes for enzymatic pathways producing biologically active secondary metabolites.

### SUMMARY OF THE INVENTION

A primary object of the invention is the use of a bacterium of the genus Microbacterium for the production of a compound endowed with antibacterial activity.

According to an embodiment, the bacterium is selected from the group consisting of Microbacterium sp., Microbacterium arborescens, Microbacterium liquefaciens, Microbacterium maritypicum and Microbacterium oxydans.

Preferably, the bacterium is Microbacterium arborescens, more particularly Microbacterium arborescens CIP 55.81T.

According to the invention, the compound endowed with antibacterial activity is extracted after culturing said bacterium.

According to an embodiment, the bacterium is cultured in a nutrient medium comprising a source of carbon, a source of nitrogen and a yeast extract.

Preferably, the bacterium is cultured at a temperature range of from about 20° C. to about 40° C.

Preferably, the bacterium is cultured at a pH of from about 6 to about 8.

Preferably, the bacterium is cultured for a period of from about 10 to about 144 hours.

According to an embodiment, the compound endowed with antibacterial activity is extracted by a liquid-liquid extraction or by a solid-liquid extraction in contact with a polar or a non-polar solvent or mixtures thereof.

A further object of the invention is the use of secondary metabolites produced by the genus Microbacterium as compounds endowed with antibacterial activity.

The present invention shows that microorganisms of the genus Microbacterium are capable of producing compounds endowed with antibacterial activity, thus a further object of the invention is to provide a method for efficiently and effectively producing such compounds endowed with antibacterial activity comprising the technical steps described above.

In accordance with the teachings of the present invention, identified and utilized as disclosed herein are bacterium strains of the genus Microbacterium which are useful for the production of compounds endowed with antibacterial activity.

In further accordance with the teachings of the present invention, disclosed herein is a method for the production of compounds endowed with antibacterial activity. This method is comprised of culturing a strain of the genus Microbacterium, whereby compounds endowed with antibacterial activity are produced thereby.

Preferably, the method of the present invention comprises culturing one of the following strains of the genus Microbacterium: Microbacterium sp., Microbacterium arborescens, Microbacterium liquefaciens, Microbacterium maritypicum and Microbacterium oxydans.

Preferably, the method of the present invention comprises culturing the strain Microbacterium arborescens, and more particularly the strain Microbacterium arborescens CIP 55.81T.

In another related aspect of the present invention, disclosed herein is a method for the production of compounds endowed with antibacterial activity produced by a strain of the genus Microbacterium. This method includes the steps of culturing a strain of the genus Microbacterium, whereby compounds endowed with antibacterial activity are produced and recovered.

### DESCRIPTION OF PREFERRED EMBODIMENTS

An embodiment of the present invention consists in providing a production process of compounds endowed with antibacterial activity which comprises culturing a bacterium strain of the genus Microbacterium on a nutrient medium, to accumulate the said compounds endowed with antibacterial activity in the nutrient medium and in the bacterium.

The microorganisms of the genus Microbacterium can be cultured in any manner which permits the growth thereof with the concurrent production of compounds endowed with antibacterial activity, as will be readily understood by one skilled in the art. In this regard, it is contemplated herein that such cultivation will be done in culture medium which also contains sources of carbon (such as glucose, starch, glycerol, sucrose and molasses), nitrogen (such as peptone, hydrolyzed derivatives of casein, meat or soja, ammonium or sodium nitrates, ammonium sulphate or phosphate, corn steep liquor, malt extract, etc.) and, if desired, mineral salts. The pH of the aqueous culture medium is controlled by buffers such as MOPS (3-(N-morpholino) propansulfonic acid), phosphoric acids salts or TRIS buffer or by continuous controlled addition of sodium, potassium or ammonium hydroxide or an inorganic acid, such as hydrochloric or sulfuric acid. The pH is maintained between about 5 and about 10, preferably between about 7 and about 9.

The temperature of such cultivation can be carried out at a temperature range of from about 20° C. to about 40° C., preferably from about 30° C. to about 35° C. for a period of from about 10 to about 144 hours, more preferably from about 72 to 96 hours depending upon the particular strain cultivated. The pH of the culture media may be from about pH 5 to about pH 10 with preferably a pH of about 7 to about 9.

The microorganisms of the genus Microbacterium may then be recovered at the end of the cultivation. Such recovery may be carried out by any suitable means therefor which are well known to those skilled in the art. As disclosed herein, such recovery is achieved by centrifugation of the culture broth so as to form a pellet containing the cells, followed by decanting of the supernatant or otherwise recovering of the cells. Although the microorganism may be used in a free form of bacterial cell, it is possible to immobilize the bacterial cell. In such an event, the bacterial cells may be immobilized while in (before the removal thereof from) the whole fermentation. After such immobilization, the fermentation broth may be easily removed therefrom by any method well known to those skilled in the art, such as by simple decanting.

Immobilization of the bacterial cells may be done by any conventional method well-known to those skilled in the art, suitable to permit the use according to the method of the present invention. Such methods include being immobilized in alginate or carrageenan gel or a high polymer film.

When the culture is terminated, the supernatant and the pellet are separated. The extraction of the compounds endowed with antibacterial activity produced by a strain of the genus Microbacterium is carried out on the pellet or the supernatant or on both with a polar solvent or a non-polar solvent, or a mixture thereof, optionally followed by a purification process which enriches the compounds endowed with antibacterial activity from the polar solvent or the non-polar solvent.

In one embodiment of this aspect of the invention, the polar solvent for extraction or purification independently include (alone or mixtures), but is not limited to, C1-C4 alcohol (e.g. methanol, ethanol, isopropanol, butanol), dimethyl sulfoxide, tetrahydrofuran, acetone, acetonitrile, or mixtures thereof.

In one embodiment of this aspect of the invention, the non-polar solvent for extraction or purification independently include (alone or mixtures), but is not limited to hexanes, heptanes, ethyl ether, ethyl acetate, petroleum ether, dichloromethane, chloroform, toluene, methyl tert-butyl ether, methyl isobutyl ketone, or mixtures thereof. Mixtures of polar and non-polar solvents can also be used, for example mixtures of chlorinated solvents and alcohols like dichloromethane methanol (DCM).

Another embodiment of the present invention consists in providing a method for production of compounds endowed with antibacterial activity which comprises culturing a strain of the genus Microbacterium on a nutrient medium, to accumulate said compounds endowed with antibacterial activity in the nutrient medium and in the bacteria.

Microbacterium, preferably Microbacterium arborescens is an example which can be most effectively used in the present invention. The physiological characteristics of this strain are as follows.

Various Physiological Properties

Optimum growth condition: The most suitable growth condition of this strain is pH 5 to 9, temperature 20° C. to 35° C.

The Microbacterium arborescens strain is cultured in or on a suitable nutrient medium at first for the production of compounds endowed with antibacterial activity. In the culture of such a strain of the present invention, an ordinary culture method is applied generally. As for the culture media, nutrient media containing a carbon source which the microorganism can assimilate, a nitrogen source which the microorganism can digest and further inorganic salts as needed are used appropriately.

Glucose, sucrose, cane sugar, molasses, starch, dextrin, cellulose, glycerin, organic acids, peptone may be used singly or in combination as the assimilable carbon source mentioned above. Organic nitrogen source such as peptone, meat extract, yeast extract, dried yeast, soybean meal, corn steep liquor, cottonseed meal, casein, soy protein hydrolysate, amino acids and urea, inorganic nitrogen compounds such as nitrates and ammonium salts may be used singly or in combination as the digestible nitrogen source.

In addition, inorganic salts such as sodium salts, potassium salts, calcium salts, magnesium salts and phosphates, heavy metal salts may be added as needed. Furthermore, micronutrients, growth promoters and precursors which promote the growth of the microorganism of the present invention and/or the production of compounds endowed with antibacterial activity substances may be added as needed, properly in the culture medium.

It is usually preferable to perform culture under aerobic conditions such as shaking culture or aeration stirring culture. Industrially, submerged aeration culture is preferable. The pH of the culture medium is preferably around neutrality. The culture temperature may be in the range of 20° C. to 40° C., but the temperature is ordinarily maintained to the range of 24° C. to 30° C., preferably around 30° C. As for the culturing time, compounds endowed with antibacterial activity are ordinarily produced and accumulated when culturing is performed for 3 to 6 days and therefore, the culturing may be preferably finished when the accumulated compounds endowed with antibacterial activity reach the maximum level.

Needless to say, these culture conditions such as the culture composition, pH of the culture medium, culture temperature, stirring rate and aeration rate may appropriately be adjusted and/or selected so that desirable results may be obtained depending on the kind of the strain to use and/or the external conditions. When foaming occurs in liquid culturing, an antifoaming agent such as silicone oil, vegetable oil and a surfactant may be used appropriately.

In order to take out the compounds endowed with antibacterial activity from the culture, the culture filtrate or the pellet are extracted with organic solvents such as acetone, ethyl acetate, butyl acetate, butanol, dichloromethane, methanol and chloroform or a mixture of these solvents, and the extract is concentrated under reduced pressure to obtain crude compounds endowed with antibacterial activity. The crude compounds endowed with antibacterial activity can be further subjected to known methods usually used for purification as column chromatography using carriers such as silica gel or reversed phase to separate and purify the compounds endowed with antibacterial activity.

### EXAMPLES

In the following, the present invention is specifically described by way of examples but the present invention is not limited to only these.

A) Qualitative Assessment of the Activity in Liquid Phase and Solid Phase

The activity of different strains of the genus Microbacterium has been qualitatively assessed against Staphylococcus aureus ATCC 13709 in solid phase and liquid phase (Table 1). Strains 1 to 8 belong to the species Microbacterium arborescens and strains 9 to 19 belong to various species of the genus Microbacterium, but the species could not be identified.

TABLE 1

| Strain | Liquid phase | Solid phase |
|---|---|---|
| 1 | X | X |
| 2 | X | X |
| 3 | X | X |
| 4 | X | X |
| 5 | X | X |
| 6 | X | X |
| 7 | X | X |
| 8 | X | X |
| 9 | X | X |
| 10 | X | X |
| 11 | X | |
| 12 | X | |
| 13 | X | |
| 14 | X | |
| 15 | | X |
| 16 | | X |
| 17 | | X |
| 18 | | X |
| 19 | | X |

It has been found that an activity is observed according to the nature of the phase wherein the culture of the Microbacterium strains was carried out.

B) Determination of the Antibacterial Activity of Compounds Produced by Microbacterium arborescens Strain CIP 55.81T

1) Preculture

100 ml of a partial yeast-peptone-glucose (YPG) medium containing 10 g/L of peptone, 5 g/L of yeast extract, was autoclaved at 121° C. for 20 minutes. After cooling down, the medium was completed by sterile MOPS solution (final concentration: 150 mM) and sterile glucose solution (final concentration: 1 g/L). The pH was adjusted to pH 7.2 with a sterile solution of KOH or HCl. The medium was inoculated with a colony of the primary Microbacterium arborescens strain CIP 55.81T, deposited in the Collection de l′Institut Pasteur (CIP) at the Biological Resource Center of Institut Pasteur, and was incubated at 30° C. for 24 h with stirring at 160 rpm. Optical Density (OD) at 600 nm was then measured by a spectrophotometer until the Microbacterium arborescens strain was at the beginning middle of its exponential growth phase (1<OD at 600 nm <3). The purity of the preculture was monitored by seeding on YPG agar. The plates were incubated at 30° C. for 48 h.

2) Cultures in Erlenmeyer Flasks

A 5000 ml flask, containing as a final volume 1000 ml sterile YPG medium was inoculated with the 100 ml of pre-culture described in paragraph 1) and incubated at 30° C. for 96 hours with stirring at 160 rpm. Initial OD at 600 nm ranged between 0.1 and 0.3. Purity of fermentation was monitored at the end of 96 hours by seeding a YPG agar. The plates were incubated at 30° C. for 48 h. The culture was centrifuged at 10,000 g for 45 min at 25° C. The supernatant and the pellet were kept at 4° C.

3) Cultures in Solid Phase

A Petri dish (10×10 cm), containing as a final volume 30 ml sterile YPG medium with 15 g/L agar, was inoculated by flooding with the 10 ml of preculture described in paragraph 1). The excess of pre-culture is then removed. The Petri dish is then incubated at 30° C. for 96 hours.

4) Extraction of the Compounds Having Antibacterial Activity (Antibacterial Agents)

Extraction of the compounds having antibacterial activity from the supernatant was carried out by extraction with dichloromethane/methanol in a 80:20 ratio. The operation was carried out 5 times. The solvent was evaporated under reduced pressure to yield the crude extracts.

Extraction of the compounds having antibacterial activity from the solid phase was carried out by covering agar pieces with a solvent such as butanol or acetone for 16 hours. The solvent was collected and concentrated to dryness under reduced pressure to give the crude extracts. 5) Antibacterial Activity of the Antibacterial Compound According to the Invention

The measures of activities were conducted on crude extracts, following the protocol recommended by the Clinical and Laboratory Standards Institute (CLSI)—Clinical and Laboratory Standards Institute (CLSI, formerly NCCLS): Dilution Antimicrobial Susceptibility Methods for Tests for Bacteria That Grow Aerobically; Approved Standard—Tenth Edition (2015). Clinical and Laboratory Standards Institute Document M07-A10.

The minimum inhibitory concentrations (MICs) of the crude extracts solubilised in DMSO are given in Table 2.

TABLE 2

| | MIC range |
|---|---|
| STRAIN | (μg/mL) |
| Staphylococcus aureus - ATCC 13709 | 5-20 |
| (Methicillin sensitive) | |
| Staphylococcus aureus MRSA - ATCC 1683 | 5-40 |
| (Methicillin resistant) | |

1-9. (canceled)

10. A method of producing compounds having antibacterial activity comprising culturing a bacterium of the genus Microbacterium to produce a compound having antibacterial activity.

11. The method according to claim 10, wherein the bacterium is selected from the group consisting of Microbacterium sp., Microbacterium arborescens, Microbacterium liquefaciens, Microbacterium maritypicum and Microbacterium oxydans.

12. The method according to claim 10, wherein the bacterium is Microbacterium arborescens or Microbacterium arborescens CIP 55.81T.

13. The method according to claim 10, wherein the compound is extracted after culturing said bacterium.

14. The method according to claim 10, wherein the bacterium is cultured in a nutrient medium comprising a source of carbon, a source of nitrogen and a yeast extract.

15. The method according to claim 10, wherein the bacterium is cultured at a temperature range of about 20° C. to about 40° C.

16. The method according to claim 10, wherein the bacterium is cultured at a pH of about 6 to about 8.

17. The method according to claim 10, wherein the bacterium is cultured for a period of about 10 to about 144 hours.

18. The method according to claim 10, wherein the compound endowed with antibacterial activity is extracted by a liquid-liquid extraction or by a solid-liquid extraction in contact with a polar or a non-polar solvent or mixtures thereof.
